(19) United States
(12) Patent Application Publication
(10) Pub. No.: US 2008/0009481 A1
(43) Pub. Date: Jan. 10, 2008

(54) Process For Making Form I Of Olanzapine

(75) Inventors: Chandiran Thakashinamoorthy, Tamil Nadu (IN); Devarajan Krishnan, Tamil Nadu (IN); Saravanan Govindaraju, Tamil Nadu (IN); Shobana Jothi, Tamil Nadu (IN)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(73) Assignee: SHASUN CHEMICALS AND DRUGS LIMITED, Tamil Nadu (IN)

(21) Appl. No.: 11/572,081

(22) PCT Filed: Jul. 13, 2005
(86) PCT No.: PCT/IN05/00239

§ 371 Date: Sep. 5, 2007

(30) Foreign Application Priority Data
Jul. 14, 2004 (IN) 678/CHE/2004

(51) Int. Cl. A61K 31/551; A61P 25/00; C07D 243/12

(52) U.S. Cl. 514/220; 540/557

(57) ABSTRACT

This invention discloses a new dihydrate polymorph of Olanzapine (hereinafter referred to as “dihydrate C”), and process for recovering anhydrous Form I of Olanzapine from this novel Dihydrate C.

### FIELD OF THE INVENTION

The present invention relates to novel dihydrate C of Olanzapine, and a process for its conversion to Form I of Olanzapine.

### BACKGROUND OF THE INVENTION

Olanzapine is an antipsychotic agent that belongs to the thienobenzodiazepine class. The chemical designation is 2-methyl-4-(4-methyl-1-piperazinyl)-1OH-thieno[2,3-b][1,5]benzodiazepine.

U.S. Pat. No. 5,736,541 discloses that Olanzapine can exist in two different crystalline polymorphs namely Form I and Form II, wherein the two polymorphs are characterized by their different X-ray power diffraction pattern and are represented by the following interplanar spacings and typical relative intensities as shown in Table—1.

TABLE 1
X-Ray powder diffraction spectrums of Form I and Form II

| | |
|---|---|
| Form II | Form I |

| d | I/I₁ | d | I/I₁ |
|---|---|---|---|
| 9.9463 | 100.00 | 10.2689 | 100.00 |
| 8.577 | 7.96 | 8.5579 | 15.18 |
| 7.4721 | 1.41 | 8.2445 | 1.96 |
| 7.125 | 6.50 | 6.8862 | 14.73 |
| 6.1459 | 3.12 | 6.3787 | 4.25 |
| 6.071 | 5.12 | 6.2439 | 5.21 |
| 4.4849 | 0.52 | 5.5895 | 1.10 |
| 5.2181 | 6.86 | 5.3055 | 0.95 |
| 5.1251 | 2.47 | 4.9815 | 6.14 |
| 4.9874 | 7.41 | 4.8333 | 68.37 |
| 4.7665 | 4.03 | 4.7255 | 21.88 |
| 4.7158 | 6.80 | 4.6286 | 3.82 |
| 4.4787 | 14.72 | 4.533 | 17.83 |
| 4.3307 | 1.48 | 4.4624 | 4.02 |
| 4.2294 | 23.19 | 4.2915 | 9.19 |
| 4.141 | 11.28 | 4.2346 | 18.88 |
| 3.9873 | 9.01 | 4.0855 | 17.29 |
| 3.7206 | 14.04 | 3.8254 | 6.49 |
| 3.5645 | 2.27 | 3.7489 | 10.64 |
| 3.5366 | 4.85 | 3.6983 | 14.65 |
| 3.3828 | 3.47 | 3.5817 | 4.04 |
| 3.2516 | 1.25 | 3.5064 | 9.23 |
| 3.134 | 0.81 | 3.3392 | 4.67 |
| 3.0848 | 0.45 | 3.2806 | 1.96 |
| 3.0638 | 1.34 | 3.2138 | 2.52 |
| 3.0111 | 3.51 | 3.1118 | 4.81 |
| 2.8739 | 0.79 | 3.0507 | 1.96 |
| 2.8102 | 1.47 | 2.948 | 2.40 |
| 2.7217 | 0.20 | 2.8172 | 2.89 |
| 2.6432 | 1.26 | 2.7589 | 2.27 |
| 2.6007 | 0.77 | 2.6597 | 1.86 |

It is well known from prior art that Form I of Olanzapine can be crystallized from methylene chloride. However, this is associated with problems of removing residual methylene chloride from the final product (limit of not more than 600 ppm) in spite of increasing drying temperatures around 70° C. for a sufficiently long time. This is surprising considering that methylene chloride has a relatively lower boiling point.

Another problem associated with obtaining Form I of Olanzapine from methylene chloride is the formation of the following impurity: 1-Chloromethyl-1-methyl-4-(2-methyl-10H-thieno-[2,3-b][1,5]benzodiazapine-piperazinium chloride (hereinafter referred to as “impurity C”), and having the following structure:

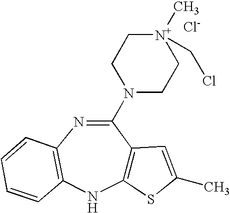

This impurity C is difficult to remove even upon multiple re-crystallizations from methylene chloride, and in fact the level of impurity increases with each re-crystallization, as this impurity C is a product formed by the reaction of Olanzapine in methylene chloride. Thus, there is a need for finding alternate ways of making pharmaceutically acceptable Form I of Olanzapine while attempting to solve the problem of impurity C formation.

### SUMMARY OF THE INVENTION

The present invention provides a novel Dihydrate C of Olanzapine.

The invention also provides for a pharmaceutical formulation comprising the novel Dihydrate C of Olanzapine.

The invention further provides a process for obtaining substantially pure Form I of Olanzapine with lower organic residues comprising drying Olanzapine Dihydrate at about 40° C. to about 70° C., until the desired Form I is obtained.

The instant invention also provides a novel process for obtaining substantially pure Form I of Olanzapine with lower organic residues comprising drying Olanzapine Dihydrate C.

### BRIEF DESCRIPTION OF THE DRAWINGS

FIG. 1. shows a typical powder X-ray diffraction pattern of Dihydrate C of Olanzapine.

Note: vertical axis represents intensity (CPS) and horizontal axis represents 2-theta degrees).

FIG. 2. shows a typical infrared spectrum of Dihydrate C of Olanzapine

FIG. 3. shows a typical differential scanning calorimetric pattern of Dihydrate C of Olanzapine

### DETAILED DESCRIPTION OF THE INVENTION

Definitions

As used herein, the term “technical grade” refers to Olanzapine having less than 5% undesired related substances. Such technical grade may contain less than about 1% undesired related substances. The term “crude” refers to form of Olanzapine having typically associated with less than 5% of undesired polymorph and/or undesired related substances. Such crude grade Olanzapine may contain less than about 1% of undesired related substances.

As used herein, term “substantially pure” refers to olanzapine associated with less than 5%, preferably less than 2% and most preferably less than 1% of the other crystalline form, as may be detected by typical spectroscopic methods. Further, “substantially pure” relates to a chemical compound which preferably contains less than about 2%, more preferably less than 1%, most preferably less than 0.5% of undesired chemical impurities, i.e. unrelated substrates, residual solvents or water.

Substantially Pure Olanzaoine Form I

Applicants have discovered that Olanzapine Form I that is substantially free from all organic residues can be prepared using an eco-friendly process, which comprises of
i) Obtaining crude Form I of Olanzapine by crystallization of technical grade Olanzapine from methylene chloride
ii) treatment of crude Form I of Olanzapine with water by stirring at 25-35 C.. for about 15 minutes to about 3 hours.
iii) subsequent filtration to obtain Olanzapine Dihydrate C
iv) drying the Olanzapine Dihydrate C, for instance in a vacuum oven, at about 40° C. to about 70° C., until the desired Form I is obtained.

Previous attempts by industry have resulted in Olanzapine Dihydrate being converted to Olanzapine form II without conversion to form I. The present inventor has successfully achieved conversion of olanzapine dihydrate to olanzapine form I, without conversion to form II. Therein lies another novelty of this invention.

Surprisingly, the present inventors found that Olanzapie dihyd can be dried to obtain Olna form 1, with low organic impurities. The starting material for the present invention (technical grade of Olanzapine) can be prepared by a variety of procedures well known to those of ordinary skill in the art. The material to be employed as starting materials in the process of this invention can be prepared by general procedure as disclosed by Chakrabati in U.S. Pat. No. 5,299,382.

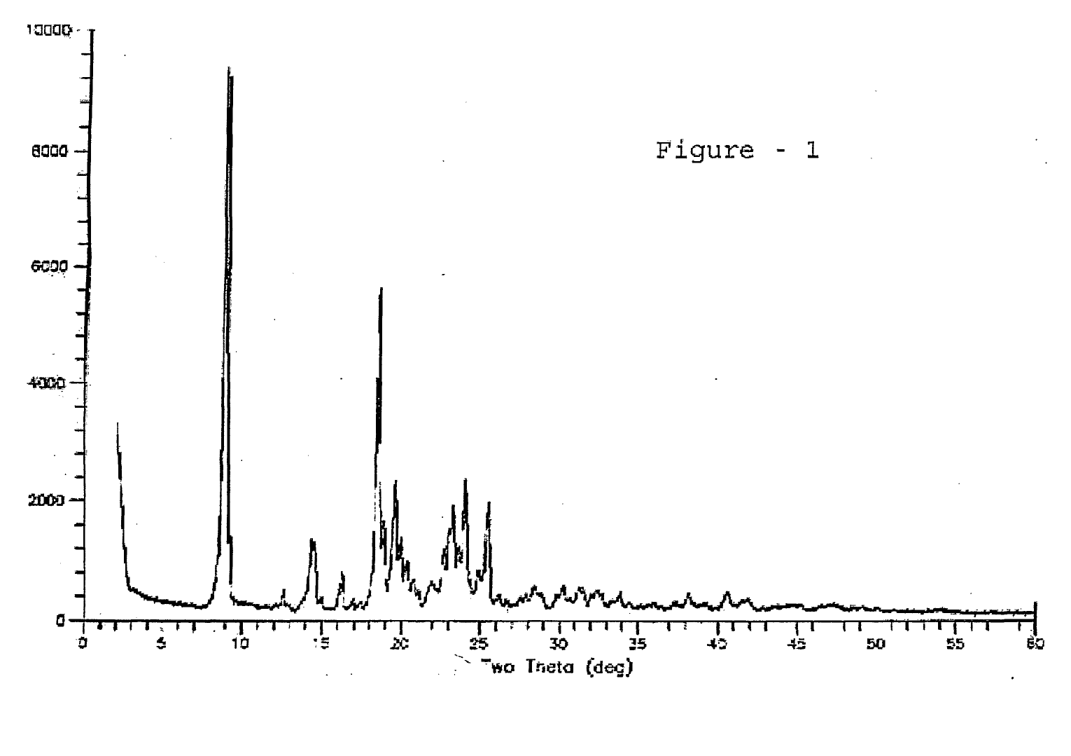

The novel dihydrate Form C of Olanzapine, as obtained in step (iii), is clearly distinguishable by powder x-ray crystallography, infrared spectroscopy, and differential scanning calorimetry. A typical x-ray power diffraction pattern is shown in FIG. 1.

Powder X-ray diffraction patterns were measured on a Shimadzu XDD1 diffractometer with Cu Kα radiation, 2-theta range 2 to 60 degree and recorded diffraction angle 2-theta, interplanar spacings d and relative intensity I/I₀ (scan speed 0.02 deg/sec, step 0.02 deg/sec, slit 1-1-0.3 mm). A typical powder X-ray diffraction pattern for this noval Olanzapine dihydrate C is given in the table 1.

TABLE 1
XRD data for Olanzapine Dihydrate-C

| 2 Theta (deg) | D (A) | I/I₁ |
|---|---|---|
| 8.88 | 9.95002 | 100 |
| 9.16 | 9.64649 | 12 |
| 12.6 | 7.01951 | 3 |
| 14.32 | 6.18002 | 11 |
| 14.48 | 6.11209 | 10 |
| 14.94 | 5.92492 | 2 |
| 16.28 | 5.44014 | 5 |
| 16.92 | 5.23577 | 2 |
| 18.42 | 4.81265 | 50 |
| 18.8 | 4.71622 | 13 |
| 19.54 | 4.53925 | 20 |
| 19.92 | 4.45351 | 11 |
| 20.34 | 4.36249 | 7 |
| 20.44 | 4.34137 | 4 |
| 20.76 | 4.27517 | 4 |
| 21.06 | 4.21494 | 3 |
| 22.02 | 4.0333 | 4 |
| 22.62 | 3.92766 | 9 |
| 22.96 | 3.87026 | 12 |
| 23.22 | 3.82751 | 15 |
| 23.58 | 3.76988 | 9 |
| 23.96 | 3.71094 | 19 |
| 24.16 | 3.68068 | 6 |
| 24.3 | 3.65979 | 4 |
| 24.4 | 3.64501 | 3 |
| 24.84 | 3.58143 | 6 |
| 25.42 | 3.50102 | 15 |
| 27.86 | 3.19969 | 2 |
| 28.38 | 3.14223 | 3 |
| 28.82 | 3.09525 | 2. |
| 29.78 | 2.99762 | 2 |
| 29.9 | 2.98587 | 2 |
| 30.24 | 2.95306 | 4 |
| 30.34 | 2.94356 | 2 |
| 31.24 | 2.86078 | 3 |
| 31.54 | 2.83425 | 2 |
| 32.64 | 2.7412 | 2 |
| 33.86 | 2.64517 | 2 |
| 38.18 | 2.35522 | 2 |
| 40.58 | 2.22129 | 3 |
| 40.70 | 2.21502 | 2 |

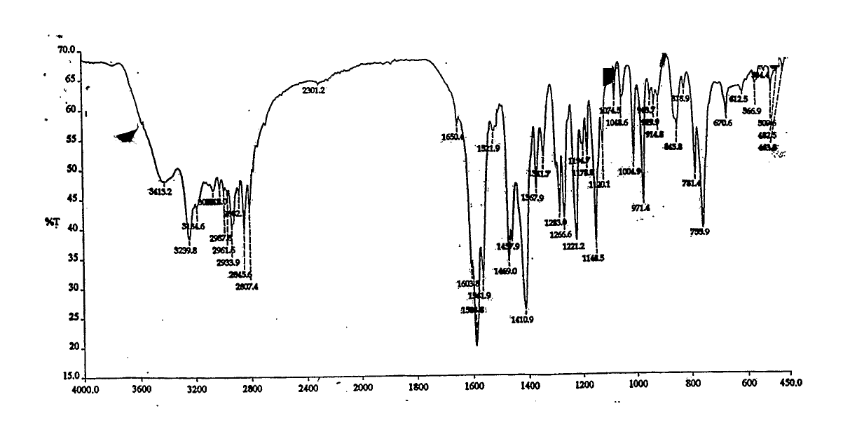

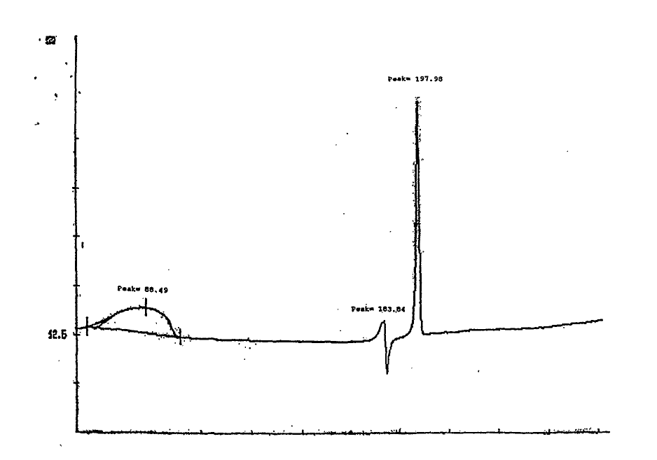

Further characterization of Olanzapine dihydrate C by infrared spectroscopy and differential scanning calorimetry is provided in FIG. 2 and FIG. 3 respectively.

The infrared (FT-IR) spectra were obtained in a KBR disk using a perkin Elmer FT-IR spectrometer spectrum one at resolution 4 cm⁻¹ from 4000 to 400 cm⁻¹. A typical FT-IR spectra of the novel Olanzapine dihydrate C showed absorptions at the following wave numbers (cm⁻¹): 3412, 3240, 3063, 2933, 2845, 2807, 1589, 1561, 1468, 1457, 1411, 1367, 1342, 1282, 1266, 1221, 1200, 1178, 1148, 1120, 1074, 1048, 1005, 971, 944, 929, 846, 818, 781, 756, 670, 509.

The differential scanning calorimetry was run on Perkin Elmer DSC 7 at a scanning rate of 10° C./min. A typical differential scanning calorimetry of this novel Olanzapine dihydrate C showed endotherm at 88.5° C. due to water loss and a endotherm at 183.4° C. with imbedded exotherm due to crystal rearrangement and another endotherm at 198.0° C.

It is to be noted that treatment of crude Olanzapine Form I with water substantially reduces the content of methylene chloride in the final product. Preferably, Olanzapine Form I contains less than 200 ppm of methlylene chloride; more preferably less than 100 ppm of methlylene chloride; and, most preferably, less than 50 ppm of methlylene chloride. Comparatively, when Olanzapine Form I is ordinarily crystallized from methylene chloride the level of residual methylene chloride in the final product may levels of around 500 ppm, and more likely above 600 ppm.

The treatment of crude Olanzapine Form I with water allows for rejection of impurity C as the impurity is water-soluble. Preferably the impurity C is less than 0.1% in the final product.

The Olanzapine dihydrate C, made by the present invention, can be administered in oral formulations to a patient suffering from following symptoms/conditions: anxiety disorders selected from the group consisting of psychoactive substance disorder, organic anxiety disorder, obsessive compulsive disorder, post traumatic stress disorder, generalized anxiety disorder, and anxiety disorder NOS, or pathologic psychological conditions wherein delusions, disorganized behavior, or anxiety are a consistent manifestation of the pathologic condition comprising administering an affective amount of the Olanzapine Dihydrate C as described previously.

Conventional pharmaceutical formulations for this novel Olanzapine dihydrate C can be prepared using conventional methods such as wet granulation. A formulation will contain known diluents/excipients/acceptable carriers. A typical formulation can be made by known methods and comprise of the Olanzapine dihydrate C, mixed with lactose, starch, talc or magnesium stearate using known methods. Especially preferred are formulations with polymer coatings on the formulation. Such coatings can be selected from hydroxypropyl methylcellulose, hydroxypropyl ethyl cellulose, poly vinyl pyrrolidone, metacrylate polymers or similar polymers known to those skilled in the art of formulation.

### EXAMPLES

The following examples are presented for illustrative purposes only and are not intended as a restriction on the scope of the invention. The polymorphic form of finally prepared crystalline olanzapine may be determined by infrared (IR) spectroscopy and X-ray powder diffraction analysis.

### Example 1

### Preparation of Crude Olanzapine Form I

Crude Olanzapine (30.0 g) is added into dichloromethane (180 ml). The reaction mixture is refluxed at 35-40° C. for a period of 15-20 min. When complete dissolution occurs, activated carbon (1.20 g) is added and the reaction mixture is then refluxed at 35-40° C. for 15 min. The solution is then filtered through hy-flo bed and the bed is washed with dichloromethane (30 ml). The filtrate is then slowly cooled to 0-5° C. and then maintained at 0-5° C. for 5 h to complete the crystallization. The content is then filtered, washed with chilled (0-5°) dichloromethane (30 ml) and dried at 70° C.-75° C. under vacuum for 12 hours (Yield 14 g) to give Olanzapine Form I. Dichloromethane content 620 ppm. Impurity C is 0.2% w/w.

### Example 2

### Preparation of Olanzapine Dihydrate C

Olanzapine Form—I (10 g) is suspended in water (30 ml) and stirred at 30 to 35° C. over a period of 30 minutes. The slurry is then filtered and washed with water (50 ml) and suck dried. The product obtained is dried at 30 to 35° C. for 24 hrs (Yield: 9.5 g). The moisture content of the product is 10.2%.

The product is examined by powder x-ray crystallography, infrared spectroscopy and differential scanning calorimetry to identify the crystal form (FIGS. 1 to 3). The product is examined by powder x-ray crystallography, infrared spectroscopy and differential scanning calorimetry to identify the crystal form (FIGS. 1 to 3).

### Example 3

### Preparation of Form I of Olanzapine

The Olanzapine Dihydrate C (10 g) is dried at 60° C. to 70° C. under vacuum for 3 to 4 hours (Yield: 8.5 g) to obtain substantially pure Olanzapine Form I. The moisture content of the product is 0.2% and dichloromethane is 112 ppm. Impurity C content is 0.08% w/w. Thus it is clear that producing Olanzapine Form I by following the process as invented by the present applicant [eg. 3] results in lesser DCM content as well as lower impurity C levels.

1. Olanzapine Dihydrate C, which has an X-ray powder diffraction pattern with the following interplanar spacings (d) in Angstroms: 9.95002, 9.64649, 7.01951, 6.18002, 6.11209, 5.92492, 5.44014, 5.23577, 4.81265, 4.71622, 4.53925, 4.45351, 4.36249, 4.34137, 4.27517, 4.21494, 4.0333, 3.92766, 3.87026, 3.82751, 3.76988, 3.71094, 3.68068, 3.65979, 3.64501, 3.58143, 3.50102, 3.19969, 3.14223, 3.09525, 2.99762, 2.98587, 2.95306, 2.94356, 2.86078, 2.83425, 2.7412, 2.64517, 2.35522, 2.22129, 2.21502.

2. Olanzapine Dihydrate C, which has the following 2 Theta (deg) values: 8.88, 9.16, 12.6, 14.32, 14.48, 14.94, 16.28, 16.92, 18.42, 18.8, 19.54, 19.92, 20.34, 20.44, 20.76, 21.06, 22.02, 22.62, 22.96, 23.22, 23.58, 23.96, 24.16, 24.3, 24.4, 24.84, 25.42, 27.86, 28.38, 28.82, 29.78, 29.9, 30.24, 30.34, 31.24, 31.54, 32.64, 33.86, 38.18, 40.58, 40.70.

3. A process for preparing Olanzapine Dihydrate C comprising the step of stirring Form I of olanzapine in an aqueous medium to get Olanzapine Dihydrate C.

4. A process for preparing substantially pure Olanzapine Form I comprising drying Olanzapine Dihydrate at a predetermined temperature until substantially pure Olanzapine Form I is obtained.

5. A process according to claim 4 wherein the temperature ranges between 40 to 70° C.

6. A process according to claim 4 wherein the Olanzapine Dihydrate is Olanzapine Dihydrate C

7. (canceled)

8. A process for preparing substantially pure Olanzapine Form I comprising:
i) treating technical grade Form I of olanzapine with water,
ii) subsequent filtration of Form I of Olanzapine to obtain Olanzapine Dihydrate C, and
iii) drying said Olanzapine Dihydrate C at a predetermined temperature until desired substantially pure Olanzapine Form I is obtained.

9. A process according to claim 8 wherein said Olanzapine Dihydrate C is dried at about 40° C. to about 70° C.

10. A process according to claim 8 wherein step (i) is carried out at a temperature between 25 to 35° C. for 15 minutes to 3 hours.

11. A process according to claim 10 wherein step (i) is carried out at a temperature between 30 to 35° C. for 30 minutes.

12. A pharmaceutical formulation comprising Olanzapine Dihydrate C as in claim 1, together with a pharmaceutically acceptable diluent or carrier therefore.

13. A method of treating a patient suffering from or susceptible to an anxiety disorder selected from the group consisting of psychoactive substance disorder, organic anxiety disorder, obsessive compulsive disorder, post traumatic stress disorder, generalized anxiety disorder, and anxiety disorder NOS, or pathologic psychological conditions wherein delusions, disorganized behavior, or anxiety are a consistent manifestation of the pathologic condition comprising administering an affective amount of the Olanzapine Dihydrate C, as in claim 1.
